(19) United States
(12) Patent Application Publication
(10) Pub. No.: US 2006/0177889 A1
(43) Pub. Date: Aug. 10, 2006

(54) Rapid responding gas sensing element

(75) Inventors: David Anvar, Sunnyvale, CA (US); David Chazan, Palo Alto, CA (US); Bhairavi Parikh, Palo Alto, CA (US); Bryan Flaherty, Half Moon Bay, CA (US); Jonathan Fay, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Aperon Biosystems Corp.

(21) Appl. No.: 11/053,253

(22) Filed: Feb. 5, 2005

(51) Int. Cl. C12Q 1/26; C12M 1/34

(52) U.S. Cl. 435/25; 435/287.1; 205/777.5

(57) ABSTRACT

A gas analysis sensing element and a method of making the sensing element is disclosed. In one embodiment, the sensing element includes cytochrome-c embedded in a sol-gel matrix. The sol-gel matrix may take the form of a thin film or a monolith. The applicants have discovered a number of parameters for creating such a sensing element, including protein concentration, sol-gel pore size, surface area for the monolith embodiment, sol-gel components, and work-up temperature.

### BACKGROUND OF THE INVENTION

1. Field of the Invention

This invention relates to sensing elements that can measure the concentration of gaseous substances.

2. General Background

Analysis of a subject's exhaled breath is a promising clinical tool, with potential application in the diagnosis and management of many conditions. For instance, changes in nitric oxide (NO) concentration in exhaled breath can indicate a change in the level of inflammation in the airway of an asthmatic, indicating an increase in the likelihood of an asthma attack. Excessive carbon monoxide (CO) can indicate hemolytic jaundice, and high levels of hydrogen can indicate carbohydrate malabsorption.

To quantify the concentration of gases, various sensors have been developed. Some of these sensors detect and measure changes in bioactive substances in response to a gaseous analyte. For instance, a sensor developed by the present inventors measures the optically-quantifiable changes in a sensing element comprised of sol-gel encapsulated cytochrome-c in response to NO. This sensor and related technology are disclosed in the following U.S. patent applications, the disclosures of which are hereby incorporated herein by reference: Ser. No. 10/334,625, filed 30 Dec. 2003, Ser. No. 10/767,709, filed 28 Jan. 2004, and U.S. Provisional Application No. 60/398,216 filed Jul. 23, 2002.

Any commercially viable trace gas sensor must have a sensing element that has a sufficiently-rapid response to the analyte. In order to achieve rapid response to trace gasses a sensor must have both a mechanism for gas to get to the “chemical transducer,” a large signal for a small number of gas molecules to be detected and good specificity of the “chemical transducer” to the molecule to be detected. Here “chemical transducer” is defined as the material component of the sensing element that allows the molecule of interest to be indirectly detected by changing the transducer's state or property, which in turn can be measured directly.

The sensing element may contain an optically active protein that specifically binds the analyte, wherein the binding event results in a change in the protein's optical absorption spectrum that can directly detected and measured.

The sensing element must have a rapid response and a large change in the state of the transducer in response to the analyte. Proteins are known for their extraordinary specificity and efficient transduction and hence if applicable are often a preferred transducer for trace analysis.

Considerations in developing a protein based gas sensor include a) the appropriate method of immobilization or encapsulation to retain activity (avoiding protein denaturation), b) choice of a host material that preserves protein activity during storage and that has a sufficient porosity to allow the sample gas to easily access the protein, c) determination of an optimum protein concentration (high enough to generate a strong response to the analyte, but not so high that it impedes transport of the sample gas), and d) the state of the protein that is most appropriate for its function.

The protein should be immobilized in a host material without loss of activity due to unfolding or rupture of peptide bonds. Each protein has a different susceptibility to denaturation and will have varying sensitivity to pH of the wet process workup of the sol-gel, the choice and concentration of solvents, the concentration of ions and or additives, and finally the workup temperature.

The host material must (i) be biocompatible, (ii) have the appropriate chemical functionality on its surface, (iii) have a sufficiently high porosity to permit rapid analyte diffusion in and out of the matrix, and (iv) be suitable to the interrogation method used for detection. To be biocompatible the host material must not have any chemical functionality that can react adversely with the immobilized protein, or decompose into products that will react adversely with the protein. In addition to chemical activity the chemical functionality (hydrophilicity or hydrophobicity for example) of the surface must not induce large enough changes in the protein conformation as to result in the loss of the protein's intrinsic functionality. Finally the host material should be porous enough to allow the trace gas to rapidly diffuse to the protein and the trace gas, or an undesired reactive byproduct of the trace gas reacting with the protein, to diffuse out of the host material. In addition if the host material pore size must be large enough for the protein to remain inside without being deformed either initially or during the post processing of the host material.

### SUMMARY OF THE INVENTION

The present invention is a gas analysis sensing element and a method of making the sensing element. In one embodiment, the sensing element includes cytochrome-c embedded in a sol-gel matrix. The sol-gel matrix may take the form of a thin film or a monolith. The applicants have discovered a number of parameters for creating such a sensing element, including protein concentration, sol-gel pore size, and surface area for the monolith embodiment.

### DETAILED DESCRIPTION

In one embodiment of the invention, cytochrome C is incorporated into a sol-gel glass with a workup concentration of methanol of about 15% by volume not to exceed about 40% by volume, a catalyst concentration of about 0.02 N (Normal) not to exceed 0.2 N, and a room temperature (˜25 C) work-up temperature not to exceed 55 C. The cytochrome C is immobilized by entrapment within a polymer network which forms around the protein after it has been mixed in the pre-polymer solution.

In this embodiment, the sol-gel should be porous and hydrophilic, so that it contains enough water and has enough constrained pore geometry to facilitate cytochrome C stability, even to temperatures up to 70 C (15 to 20 degrees higher than the protein's unfolding temperature in solution as reported in the literature). The support matrix of this embodiment has a pore network with a pore width of 3.5 to 5.8 nm and a surface area of about 550 to 650 m2/g.

The optimum protein concentration is determined by the trace gas concentration to be detected, the aspect ratio of the sensor matrix, the mean pore width of the matrix, and the physical method of interrogation (for example: optical absorption spectroscopy, fluorescence, chemiluminescence, coulometric, potentiometric, impedance, piezoelectric etc.).

For trace analysis, maximum protein concentration is rarely the optimum especially as the total analyte concentration is near or on the order of the protein concentration. The optimum concentration is also a function of the host matrix aspect ratio because diffusion in a porous medium can be path length dependant if long-range connectivity is limited beyond a percolation threshold. In addition, especially when the protein size is on the order of the pore width, the protein concentration can limit the transport of the analyte.

There is an optimum concentration of protein, and this concentration is a function of pore width, the tortuosity of the medium, the size of the protein and the colligitive properties of the protein during the formation of the polymer network.

In addition, inappropriate concentrations of protein can effect the level of background and signal to noise ratio. In the case of absorption spectroscopy for example, a too-low concentration will result in very high background light level and a small signal, whereas a too-high concentration will result in a poor signal to noise ratio and other potential problems associated with the need to increase the light source ouput, like temperature control, photo-bleaching, non-linear responses to analyte concentration, just to name a few.

As explained below, an optimum final xerogel concentration for a cytochrome-c/ sol-gel sensing element would in the approximate range of 20-27 mg/ml.

The sensing element can take the form of a thin film, and in one embodiment this film may be about 600 nm thick. In this embodiment, the cytochrome C concentration in the sol solution may be dropped below about 1 mM (about 12 mg/ml). When one takes into account the shrinkage of sol-gels (approximately a factor of 2.2 per dimension), and given the thin films only shrink normal to the surface, the concentrations come to about 2.2 mM or 26.4 mg/ml of cytochrome c in the xerogel film.

In another embodiment, the sol-gel make take the form of a monolith. Such a monolith may have a peak optical density of about 2.8 absorbance units at 400 nm and respond quickly (<1 min) to concentrations below 50 ppb nitric oxide. However when the concentration of cytochrome c is increased to 0.46 mM or 5.7 mg/ml the response rates and magnitude of response decrease by about 10 times. The monolith has a pore network with a pore width of 3.5 to 5.8 nm and a surface area of about 550 to 650 m2/g. When one takes into account the shrinkage of the monolith during curing and drying, the fast responding monoliths have an approximate cytochrome c concentration of (2.2)³×0.092=0.98 mM or (2.2)³×1.1=11.71 mg/ml. These concentrations are near to but not the maximum concentration of cytochrome c allowable for a fast responding monolith. With this in mind the maximum allowable concentrations of cytochrome c in fast responding thin films and monoliths trace gas sensors are both close to 2 mM or approximately 25 mg/ml in the final xerogel state.

A key benefit of diluting cytochrome c concentration is that in going from high concentration to low concentration the protein goes to a slightly more unfolded state also referred to in the literature as the molten globule state. In the molten globule state one of the axial ligands of the porphyrin is further removed on average from the iron center, probably resulting in an opening of the protein up for more easy access to nitric oxide binding. It is observed that the same process parameters which yield a more folded protein also decrease the responsivity of the sensor. Increasing concentration of protein to 5× and 10× concentration results in a more folded state of the protein and at the same time it is observed that the rate of response to nitric oxide decreases.

In summary, relatively low protein concentrations are critical, because at higher concentrations the greater colligative properties of the protein yield a more clogged network of pores and less protein-to-glass interaction. The former slows down transport and the latter can slow down the rate of binding of cytochrome c with nitric oxide.

One skilled in the art will appreciate that the present invention can be practiced by other than the preferred embodiments, which are presented for purposes of illustration and not of limitation.

We claim:

1. A gas sensing element, comprising:
a. a xerogel, said xerogel having a pore size in the range of 3.0 nm to 6.0 nm; and
b. a bioactive molecule embedded in said xerogel.

2. The sensing element according to claim 1, wherein the sensing element is a monolith.

3. The sensing element according to claim 1, wherein the sensing element is a thin film.

4. The sensing element according to claim 1, wherein said bioactive molecule is a protein.

5. The sensing element according to claim 3, wherein said bioactive molecule is cytochrome-c.

6. The sensing element according to claim 1, wherein said xerogel has a surface area between 550 m2/g to 650 m2/g.

7. The sensing element according to claim 3, wherein said xerogel has a protein concentration of between 1-40 mg/ml.

8. The sensing element according to claim 4, wherein said xerogel has a protein concentration of between 1-40 mg/ml.

9. The sensing element according to claim 5, wherein said xerogel has a protein concentration of between 1-40 mg/ml.

10. A gas sensing element, comprising:
a. a xerogel; and
b. a protein embedded in said xerogel, said protein having a concentration of between 1-40 mg/ml.

11. The sensing element according to claim 9, wherein the sensing element is a monolith.

12. The sensing element according to claim 9, wherein said protein is cytochrome-c.

13. The sensing element according to claim 9, wherein said xerogel has a surface area between 550 m2/g to 650 m2/g.

14. The sensing element according to claim 10, wherein said xerogel has a surface area between 550 m2/g to 650 m2/g.

15. The sensing element according to claim 11, wherein said xerogel has a surface area between 550 m2/g to 650 m2/g.

16. A gas sensing element, comprising:
a. a xerogel, said xerogel having a surface area between 550 m2/g to 650 m2/g; and
b. a bioactive molecule embedded in said xerogel.

17. The sensing element according to claim 15, wherein the sensing element is a monolith.

18. The sensing element according to claim 15, wherein said bioactive molecule is a protein.

19. The sensing element according to claim 5, wherein said bioactive molecule is cytochrome-c.

20. A method of measuring a trace gas in exhaled breath, comprising:
a. capturing exhaled breath from a subject;
b. allowing said exhaled breath to interact with a sensing element according to claim 1; and
c. measuring a trace gas concentration in said exhaled breath based upon said step of allowing said exhaled breath to interact with a sensing element according to claim 1.

21. A method of measuring a trace gas in exhaled breath, comprising:
a. capturing exhaled breath from a subject;
b. allowing said exhaled breath to interact with a sensing element according to claim 9; and
c. measuring a trace gas concentration in said exhaled breath based upon said step of allowing said exhaled breath to interact with a sensing element according to claim 9.

22. A method of measuring a trace gas in exhaled breath, comprising:
a. capturing exhaled breath from a subject;
b. allowing said exhaled breath to interact with a sensing element according to claim 15; and
c. measuring a trace gas concentration in said exhaled breath based upon said step of allowing said exhaled breath to interact with a sensing element according to claim 15.
